(19) United States
(12) Patent Application Publication
(10) Pub. No.: US 2023/0225743 A1
(43) Pub. Date: Jul. 20, 2023

(54) HEMOSTASIS BAND AND TOOL FOR APPLYING A CONSTANT PRESSURE TO A CANNULATION SITE

(71) Applicants: FRESENIUS MEDICAL CARE DEUTSCHLAND GMBH, Bad Homburg (DE); FRESENIUS MEDICAL CARE AG & CO. KGAA, Bad Homburg (DE); FRESENIUS MEDICAL CARE HOLDINGS, INC., Waltham, MA (US)

(72) Inventors: Pascal KOPPERSCHMIDT, Dittelbrunn (DE); Min HU, Xuhui, Shanghai (CN); Ralf MUELLER, Bad Homburg (DE); Pia DANIEL, Bodman (DE); David HANNES, Frankfurt (DE); Karen Granger BUTLER, Framingham, MA (US); Nadine KUBAT, Frankfurt (DE); Pari DATTA, Upper Cambourne, Cambridgeshire (GB); Ben KUBLER, Great Cambourne, Cambridgeshire (GB)

(73) Assignees: FRESENIUS MEDICAL CARE DEUTSCHLAND GMBH, Bad Homburg (DE); FRESENIUS MEDICAL CARE AG & CO. KGAA, Bad Homburg (DE); FRESENIUS MEDICAL CARE HOLDINGS, INC., Waltham, MA (US)

(21) Appl. No.: 18/007,101

(22) PCT Filed: Jul. 30, 2020
(86) PCT No.: PCT/CN2020/105664

§ 371 Date: Jan. 27, 2023

(51) Int. Cl. A61B 17/132; A61B 90/00

(52) U.S. Cl. CPC A61B 17/1327; A61B 90/08; A61B 2090/0811

(57) ABSTRACT

The present invention relates to a hemostasis band, comprising a band, preferably made from an elastic material, a pressure focusing element for focusing pressure generated by the band to hold a cannula in place at a cannulation point, wherein the band can be arranged in a loop and the size of the loop formed by the band is adjustable to at least one defined size by means of an external tool, such as a torque driver. Another aspect of the invention relates to a system of a hemostasis band and an external tool.

The present invention relates to a hemostasis band for applying a defined constant pressure to a cannulation site to prevent undesired blood flow after cannulation.

Especially for weak or elderly patients, it often proves impossible to manually press a hemostasis swab to a cannulation site until blow flow stops.

Thus, in many cases, medical personnel have to manually press the hemostasis swab to the cannulation site of such patients. Because it can easily take up to 10 minutes or more before blood flow stops, a lot of time is taken just by medical personnel holding swabs to patient’s arms.

To alleviate this problem, conventionally hemostasis bands are used to affix the swab to a patient’s arm.

Such conventional hemostasis bands, however, do not reproducibly apply a defined pressure to the patient’s arm.

Thus, in many cases the hemostasis bands apply too much pressure to the cannulation site causing patient discomfort or risks to patient safety. Especially in patients having an arteriovenous fistula or an arteriovenous shunt / graft too much pressure may cause these structures to be damaged.

Alternatively, if too little pressure is applied, the pressure is insufficient for efficient hemostasis.

It is the object of the present invention to alleviate or even completely resolve the technical problems of the prior art. In particular, it is an object of the present invention to provide a hemostasis band for reproducibly applying a constant pressure to a cannulation site.

This object is achieved by a hemostasis band comprising the features of claim 1. Further advantageous embodiments of the invention are the subject-matter of the dependent claims.

A hemostasis band according to the present invention comprises
a band, preferably made from an elastic material,
a pressure focusing element for focusing pressure generated by the band to support hemostasis after cannulation at a cannulation point,
wherein the band can be arranged in a loop and the size of the loop formed by the band is adjustable to at least one defined size, preferably by means of an external tool.

The band can be arranged in a loop but can also be open, e.g. be attached to the pressure focusing element only at one end. The band can also comprise multiple sections that each are attached to the pressure focusing element, similarly to a wrist band of a wrist watch.

When the hemostasis band is used, the band is placed around a patient’s arm and tightened or loosened by means of the tool as required by this particular patient.

An external tool is used, i.e. the tool is not part of the hemostasis band itself, but separate. This means, the tool can be separately calibrated and tested etc., independent from the hemostasis band.

This allows to manufacture the hemostasis band easily and cheaply, so the hemostasis band can be a disposable product. Of course, the hemostasis band can also be configured as a multi-use tool and thus reused.

Preferably, the band is made of a flexible and / or elastic material, e.g. silicone. If the band is placed around a patient’s arm, it is stretched and thereby generates pressure that is applied to the patient’s arm, for example, to press a swab onto a cannulation site. The swab is preferably arranged between the pressure focusing element and the patient’s body when the hemostasis band is in use.

According to an advantageous embodiment of the invention, the pressure focusing element further comprises an adjusting element by means of that the size of the loop is variable.

The adjusting element preferably is configured to interact with the external tool to adjust the size of the loop formed by the band.

For example, the external tool may be a screw driver or a torque driver and the adjusting element can have a recess configured to at least partially receive the tool.

If the tool is rotated in the recess in a clock-wise or counter-clockwise direction, the loop formed by the band can be tightened or loosed as desired.

In other words, the size of the loop formed by the band can be varied/ adjusted in a defined and controlled fashion by the interaction of the tool and the adjusting element.

Preferably, the adjusting element and / or the external tool comprises at least one marking that defines a certain relative position of the external tool and the adjusting element, wherein the relative position of the external tool and the adjusting element sets the size of the loop formed by the band to a defined size.

In other words, the at least one marking encodes a certain setting of the adjusting element that corresponds to a certain defined size of the loop. Once the size is set and the tool is removed from the adjusting element, the band is locked in place, i.e. the size that has been set remains constant until the size is varied again by use of the tool. There is no undesired slipping of the band or of ends of the band, instead, the size of the loop can only be varied in a controlled fashion by use of the tool and the adjusting element.

The at least one marking can be color-coded, e.g. a green marking can indicate a loose fit and red marking can indicate a tight fit. The marking can have any desired form and can be e.g. a symbol, a line, a number, a letter or even a word or a patient’s name.

It has proved advantageous in practice if the adjusting element and / or the external tool comprises multiple markings indicating multiple relative positions between the external tool and the adjusting element that each set the size of the loop formed by the band to a different defined size.

The size of the loop can be varied in a continuous or step-wise fashion.

The multiple markings can take the form of a scaling and may in addition be color-coded. For example, three markings may be present e.g. a green marking indicating a loose fit, a yellow marking indicating a medium fit and a red marking indicating a tight fit.

It has further proved advantageous, if the adjusting element comprises a recess configured to receive an external tool for adjusting the size of the loop formed by the band. The interaction between the tool and the adjusting element can be via form-fit and / or friction fit.

The adjusting element can be configured to change the size of the loop in response to a rotation of the tool inserted into the recess of the adjusting element. This configuration is especially advantageous if the tool is a screw driver or a torque driver.

In this case, the adjusting element preferably transforms a rotational movement of the external tool, e.g. the torque driver, into a translational movement of at least one end of the band relative to the other end of the band. If the tool is rotated in a first direction, the loop is tightened and if the tool is rotated in a second direction, the loop is loosened.

In an advantageous embodiment, the band is continuous along its length from a first end of the band that is attached to the pressure focusing element and/ or the adjusting element and a second end of the band that is attached to the pressure focusing element and/ or the adjusting element.

In other words, the band forms a continuous loop without any gaps from a first end of the pressure focusing element and/ or the adjusting element to a second end thereof.

In order to attach the hemostasis band to a patient’s arm, the size of the loop is adjusted so that the patient’s hand can reach through the loop. The hemostasis band is then tightened to smugly fit onto the patients forearm so that a pressure that is optimal for that particular patient is applied by the band onto the patient’s arm. To remove the band again from the patient’s arm, the size of the loop is increased again to allow the patient to retract his / her hand from the loop.

To improve the pressure focusing function of the pressure focusing element, the pressure focusing element may comprise a protrusion or hump on the side facing the patient’s body when the hemostasis band is arranged around at patient’s arm. The protrusion or hump helps to apply the pressure in a precise, targeted and localized fashion to a desired site, e.g. a cannulation site.

Preferably, the adjusting element and / or the pressure focusing element is configured to partially or completely surround the ends of the band. In other words, the ends of the band are not visible to a user and don’t obstruct access of the tool to the adjusting element. Preferably, the size of the loop can be varied stepwise or continuously by changing the relative position of the ends of the band.

Another aspect of the invention relates to a system comprising a hemostasis band, preferably a hemostasis band according to the present invention, and an external tool, wherein the hemostasis band comprises an adjusting element by means of that the size of a loop formed by the band can be varied; and the external tool is configured to actuate the adjusting element to vary the size of the loop formed by the band.

Preferably, the adjusting element comprises a recess configured to at least partially receive the external tool for adjusting the size of the loop formed by the band and preferably the adjusting element transforms a rotational movement of the external tool into a translational movement of at least one end of the band relative to the other end of the band.

It has proved advantageous if the adjusting element and / or the external tool comprises at least one marking that defines a certain relative position of the external tool and the adjusting element, wherein the relative position of the external tool and the adjusting element sets the size of the loop formed by the band to a defined size.

The marking can take any desired form (line, number, symbol, letter etc.) and may alternatively or additionally be color-coded.

It is also possible that the adjusting element and / or the external tool comprises multiple markings indicating multiple relative positions between the external tool and the adjusting element that each set the size of the loop formed by the band to a different defined size.

The multiple markings may form a scaling. Alternatively or additionally the multiple markings can be color-coded.

Regarding the at least one marking or the multiple markings present on the tool or the adjusting element it is preferable if marking / markings is /are present on a proximal end of the tool or the adjusting element when viewed by a user.

The proximal end of the tool or the adjusting element is the end that is closer to the user in the viewing direction of the user. This has the advantage that the marking / markings is / are always easily visible by the user handling the hemostasis band and tool.

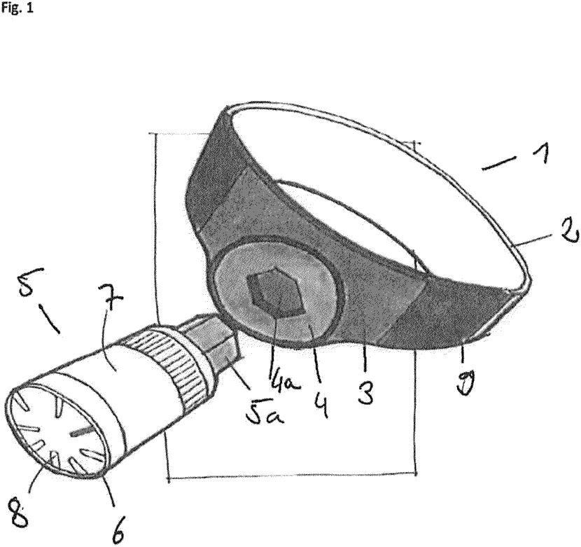

Further features, advantages and effects of the present invention become apparent from the following description of an embodiment of the invention under reference to FIG. 1.

FIG. 1 shows a hemostasis band 1 comprising a band 2 forming a loop, a pressure focusing element 3 and an adjusting element 4.

The band 2 is made of a flexible elastic material and forms a continuous loop from one end that is attached to the pressure focusing element 3 to the other end that is attached to the pressure focusing element 3.

The ends of the band 2 are movably contained in the pressure focusing element 3 and thus are not visible in FIG. 1. The pressure focusing element 3 is made of a relatively stiff polymer. Arranged at and partially embedded in the focusing element 3 is the adjusting element 4. The adjusting element can be made of metal to improve the durability of the adjusting element 4.

The adjusting element 4 comprises a recess 4a configured to receive a corresponding actuation part 5a of the external tool 5. In this embodiment, the recess 4a has a hexagonal shape and the tool 5 is a torque driver.

On a proximal end 6 of the tool 5, in particular of a gripping portion 7 of the tool 5, multiple markings 8 are provided to indicate how far the torque driver 5 has to be turned to tighten or loosen the fit of the loop formed by the band 2 to a desired degree.

To ensure a safe and comfortable fit of the hemostasis band 1 around a patient’s arm, the band also comprises stiffening portions 9 arranged between the pressure focusing element 3 and the band 2.

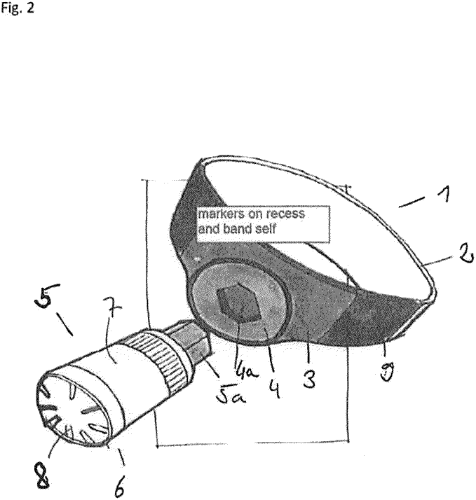

According to another embodiment shown in FIG. 2, the markings can be present on the adjusting element 4 and / or the recess 4a and the band 2.

1. Hemostasis band, comprising
– a band, preferably made from an elastic material,
– a pressure focusing element for focusing pressure generated by the band to support hemostasis after cannulation,
– wherein the band can be arranged in a loop and the size of the loop formed by the band is adjustable to at least one defined size, preferably by means of an external tool.

2. Hemostasis band according to claim 1, wherein the pressure focusing element further comprises an adjusting element by means of that the size of the loop is variable.

3. Hemostasis band according to claim 1, wherein the adjusting element is configured to interact with the external tool to adjust the size of the loop formed by the band.

4. Hemostasis band according to claim 1, wherein the adjusting element and / or the external tool comprises at least one marking that defines a certain relative position of the external tool and the adjusting element, wherein the relative position of the external tool and the adjusting element sets the size of the loop formed by the band to a defined size.

5. Hemostasis band according to claim 4, wherein the adjusting element and / or the external tool comprises multiple markings indicating multiple relative positions between the external tool and the adjusting element that each set the size of the loop formed by the band to a different defined size.

6. Hemostasis band according to claim 1, wherein the adjusting element comprises a recess configured to receive an external tool for adjusting the size of the loop formed by the band.

7. Hemostasis band according to claim 6, wherein the adjusting element is configured to change the size of the loop in response to a rotation of the tool inserted into the recess of the adjusting element.

8. Hemostasis band according to claim 1, wherein the adjusting element transforms a rotational movement of the external tool into a translational movement of at least one end of the band relative to the other end of the band.

9. Hemostasis band according to claim 1, wherein the band is continuous along its length from a first end of the band that is attached to the pressure focusing element and/ or the adjusting element and a second end of the band that is attached to the pressure focusing element and/ or the adjusting element.

10. Hemostasis band according to claim 1, wherein the adjusting element and / or the pressure focusing element is configured to partially or completely surround the ends of the band.

11. Hemostasis band according to claim 1, wherein the size can be varied stepwise or continuously.

12. System comprising a hemostasis band, preferably a hemostasis band according to claim 1, and an external tool, wherein
the hemostasis band comprises an adjusting element by means of that the size of a loop formed by the band can be varied;
the external tool is configured to actuate the adjusting element to vary the size of the loop formed by the band.

13. System according to claim 12 wherein the adjusting element comprises a recess configured to at least partially receive the external tool for adjusting the size of the loop formed by the band and preferably the adjusting element transforms a rotational movement of the external tool into a translational movement of at least one end of the band relative to the other end of the band.

14. System according to claim 12, wherein the adjusting element and / or the external tool comprises at least one marking that defines a certain relative position of the external tool and the adjusting element, wherein the relative position of the external tool and the adjusting element sets the size of the loop formed by the band to a defined size.

15. System according to claim 12. wherein the adjusting element and / or the external tool comprises multiple markings indicating multiple relative positions between the external tool and the adjusting element that each set the size of the loop formed by the band to a different defined size.

16. System according to claim 12, wherein the at least one marking that defines a certain relative position of the external tool and the adjusting element is present on a proximal end of the tool when viewed by a user.

17. System according to claim 12, wherein the tool is a torque driver or screw driver.
